(19) United States
(12) Patent Application Publication
(10) Pub. No.: US 2011/0123536 A1
(43) Pub. Date: May 26, 2011

(54) NOVEL HUMAN ANTI-R7V ANTIBODIES AND USES THEREOF

(75) Inventors: Jean Claude Chermann, Cassis (FR); Camille Haslin, Marseille (FR)

(73) Assignee: URRMA R&D

(21) Appl. No.: 12/531,843

(22) PCT Filed: Mar. 19, 2008
(86) PCT No.: PCT/EP08/53317

§ 371 Date: Apr. 13, 2010

Related U.S. Application Data
(60) Provisional application No. 60/896,359, filed on Mar. 22, 2007.

(51) Int. Cl. A61K 39/395; C07K 16/08; C07H 21/04; C12N 15/63; C12N 5/10; C12N 1/21; C12N 1/19; C12P 21/02; A61P 31/18

(52) U.S. Cl. 424/139.1; 530/324; 530/387.9; 536/23.53; 435/320.1; 435/348; 435/325; 435/358; 435/252.3; 435/254.2; 435/69.6

(57) ABSTRACT

The present application relates to novel human antibodies capable of binding specifically to the R7V epitope of HIV. These antibodies have all human CDR and are capable of specifically neutralizing all strains of HIV, including escape mutants. These antibodies are useful for the treatment of HIV infection, especially in patients in failure of HAART.

The present invention relates to novel human antibodies capable of binding specifically to the R7V epitope of HIV. These antidodies have all human CDR and are capable of specifically neutralizing all strains of HIV, including escape mutants. They are useful for the treatment of HIV infection, especially in patients in failure of HAART.

### BACKGROUND OF THE INVENTION

HIV infection is still a public health pandemic. Whereas drug therapies allow to limit HIV replication and virulence after infection, a preventive or curative treatment is not available as yet. Furthermore, the highly active antiretroviral therapy (HAART), leading to several side effects and to the emergence of drug-resistant viruses, beside the diminution of AIDS, underscores the need for additional therapeutic approaches against HIV. However, some HIV infected patients designed as non-progressor do not develop AIDS disease after 10, 15 of more years of infection, demonstrating that HIV diseases could be delayed by various ways like the presence of attenuated viruses', defective viruses², HIV coreceptors mutations^{3, 4}, or neutralizing antibodies⁵. Conventional envelope-based antibody inducing vaccines have all shown their limits on account of the high mutation rate of the virus and their poor immunogenicity. In a previous study⁶, we demonstrated the potentialities of broad spectrum neutralizing anti-R7V antibodies purified from non-progressor sera. These immunoglobulins were directed against a cellular epitope called R7V (RTPKIQV amino-acids sequence) derived from the β2-microglobulin and incorporated in HIV's coat during budding^{7, 8}. Our aim was to produce a recombinant anti-R7V antibody after the isolation of the corresponding gene from B-lymphocytes of non-progressor patients through a baculovirus vector.

The baculovirus technology allows the production and secretion of correctly assembled and glycosylated immunoglobulins⁹. These recombinant antibodies present all the functional properties of the parental immunogloblins^{10, 11} and exhibits efficient effector functions such as the binding (i) of complement component Clq^{12, 13} or C3¹⁴ and (ii) IgG Fc receptors required to induce antibody direct cellular cytotoxicity^{15, 16, 13}.

In connection with the invention, we constructed a recombinant antibody directed against the cellular epitope R7V acquired by HIV during the viral budding. The c-DNAs encoding the variable regions of the anti-R7V antibody have been cloned from B lymphocytes of a non-progressor patient. Two transfer vectors containing complete coding sequences for heavy and light chains of this antibody were constructed and a recombinant baculovirus was generated by a double recombination between baculovirus DNA and the two transfer vectors. Insect cells infected with this baculovirus produced a complete human anti-R7V immunoglobulin. We have shown that our recombinant antibody, specific to the R7V peptide, recognizes and neutralizes all clades of HIV1 including resistant viruses, which opens new perspectives in anti-HIV therapy.

### DESCRIPTION

Thus, according to a first embodiment, a subject of the present invention is an isolated antibody, or one of its functional fragments, said antibody or one of its said fragments being capable of binding specifically to the R7V epitope (RTPKIQV—SEQ ID No 11) and capable of neutralizing HIV strains, wherein it comprises:

i) a light chain comprising the complementarity determining regions CDRs comprising amino acid sequence SEQ ID No 1 (QSVLYSSNNKNY), SEQ ID No 2 (WAS) and SEQ ID No 3 (QQYYSTPQT), or CDRs which sequences have at least 80%, preferably 90% identity, after optimum alignment, with the sequence SEQ ID No 1, 2 or 3, and
ii) a heavy chain comprising the CDRs comprising amino acid sequence SEQ ID No 6 (GGSISSYY), SEQ ID No 7 (IYYSGST) and SEQ ID No 8 (ARGRSWFSY), or CDRs whose sequence have at least 80%, preferably 90% identity, after optimum alignment, with the sequence SEQ ID No 6, 7 and 8.

In the present description, the terms polypeptides, polypeptide sequences, peptides and proteins attached to antibody compounds or to their sequence are interchangeable.

It must be understood here that the invention does not relate to the antibodies in natural form, that is to say they are not in their natural environment but that they have been able to be isolated or obtained by purification from natural sources, or else obtained by genetic recombination, or by chemical synthesis, and that they can then contain unnatural amino acids as will be described further on.

By CDR region or CDR, it is intended to indicate the hypervariable regions of the heavy and light chains of the immunoglobulins as defined by Kabat et al. (Kabat et al., Sequences of proteins of immunological interest, 5th Ed., U.S. Department of Health and Human Services, NIH, 1991, and later editions). 3 heavy chain CDRs and 3 light chain CDRs exist. The term CDR or CDRs is used here in order to indicate, according to the case, one of these regions or several, or even the whole, of these regions which contain the majority of the amino acid residues responsible for the binding by affinity of the antibody for the antigen or the epitope which it recognizes.

By “percentage of identity” between two nucleic acid or amino acid sequences in the sense of the present invention, it is intended to indicate a percentage of nucleotides or of identical amino acid residues between the two sequences to be compared, obtained after the best alignment (optimum alignment), this percentage being purely statistical and the differences between the two sequences being distributed randomly and over their entire length. The comparisons of sequences between two nucleic acid or amino acid sequences are traditionally carried out by comparing these sequences after having aligned them in an optimum manner, said comparison being able to be carried out by segment or by “comparison window”. The optimum alignment of the sequences for the comparison can be carried out, in addition to manually, by means of the local homology algorithm of Smith and Waterman (1981) [Ad. App. Math. 2:482], by means of the local homology algorithm of Neddleman and Wunsch (1970) [J. Mol. Biol. 48: 443], by means of the similarity search method of Pearson and Lipman (1988) [Proc. Natl. Acad. Sci. USA 85:2444), by means of computer software using these algorithms (GAP, BESTFIT, FASTA and TFASTA in the Wisconsin Genetics Software Package, Genetics Computer Group, 575 Science Dr., Madison, Wis., or else by BLAST N or BLAST P comparison software).

The percentage of identity between two nucleic acid or amino acid sequences is determined by comparing these two sequences aligned in an optimum manner and in which the nucleic acid or amino acid sequence to be compared can comprise additions or deletions with respect to the reference sequence for an optimum alignment between these two sequences. The percentage of identity is calculated by determining the number of identical positions for which the nucleotide or the amino acid residue is identical between the two sequences, by dividing this number of identical positions by the total number of positions in the comparison window and by multiplying the result obtained by 100 in order to obtain the percentage of identity between these two sequences.

For example, it is possible to use the BLAST program, “BLAST 2 sequences” (Tatusova et al., “Blast 2 sequences—a new tool for comparing protein and nucleotide sequences”, FEMS Microbiol Lett. 174:247-250) available on the site http://www.ncbi.nlm.nih.gov/gorf/b12.html, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program.

By amino acid sequence having at least 80%, preferably 85%, 90%, 95% and 98% identity with a reference amino acid sequence, those having, with respect to the reference sequence, certain modifications, in particular a deletion, addition or substitution of at least one amino acid, a truncation or an elongation are preferred. In the case of a substitution of one or more consecutive or nonconsecutive amino acid(s), the substitutions are preferred in which the substituted amino acids are replaced by “equivalent” amino acids. The expression “equivalent amino acids” is aimed here at indicating any amino acid capable of being substituted with one of the amino acids of the base structure without, however, essentially modifying the biological activities of the corresponding antibodies and such as will be defined later, especially in the examples.

These equivalent amino acids can be determined either by relying on their structural homology with the amino acids which they replace, or on results of comparative trials of biological activity between the different antibodies capable of being carried out.

By way of example, mention is made of the possibilities of substitution capable of being carried out without resulting in a profound modification of the biological activity of the corresponding modified antibody. It is thus possible to replace leucine by valine or isoleucine, aspartic acid by glutamic acid, glutamine by asparagine, arginine by lysine, etc., the reverse substitutions being naturally envisageable under the same conditions.

The antibodies according to the present invention are preferably fully human monoclonal antibodies or functional fragments thereof.

In a particular embodiment, the antibody of the invention is featured by a light chain comprising an amino acid sequence having at least 80%, preferably 90% identity, after optimum alignment, with the amino acid sequence displayed in FIG. 3B—SEQ ID No 4 or a light chain encoded by a nucleotidic sequence comprising the sequence as depicted in FIG. 3A—SEQ ID No 5 or a sequence having at least 80%, preferably 90% identity, after optimum alignment, with SEQ ID No 5.

In another particular embodiment, the antibody of the invention is featured by a heavy chain a heavy chain comprising an amino acid sequence having at least 80%, preferably 90% identity, after optimum alignment, with the amino acid sequence displayed in FIG. 3D—SEQ ID No 9 or a heavy chain encoded by a nucleotidic sequence comprising the sequence as depicted in FIG. 3C—SEQ ID No 10 or a sequence having at least 80%, preferably 90% identity, after optimum alignment, with SEQ ID No 10. In still another embodiment, the antibody according to the invention comprises a light chain comprising the amino acid sequence displayed in FIG. 3B—SEQ ID No 4 or encoded by a nucleotidic sequence comprising the sequence as depicted in FIG. 3A-SEQ ID No 5 and a heavy chain comprising the amino acid sequence displayed in FIG. 3D—SEQ ID No 9 or encoded by a nucleotidic sequence comprising the sequence as depicted in FIG. 3C—SEQ ID No 10.

By functional fragment of an antibody according to the invention, it is intended to indicate in particular an antibody fragment, such as Fv, scFv (sc for single chain), Fab, F(ab′)₂, Fab′, scFv-Fc fragments or diabodies, or any fragment of which the half-life time would have been increased by chemical modification, such as the addition of poly(alkylene) glycol such as poly(ethylene) glycol (“PEGylation”) (pegylated fragments called Fv-PEG, scFv-PEG, Fab-PEG, F(ab′)₂—PEG or Fab′-PEG) (“PEG” for Poly(Ethylene) Glycol), or by incorporation in a liposome, said fragments having CDRs of sequence SEQ ID No. 1, 2, 3, 6, 7 and 8 according to the invention, and, especially, in that it is capable of neutralizing HIV strains.

Preferably, said functional fragments will be constituted or will comprise a partial sequence of the heavy or light variable chain of the antibody from which they are derived, said partial sequence being sufficient to retain the same specificity of binding. Preferably, these functional fragments will be fragments of Fv, scFv, Fab, F(ab′)₂, F(ab′), scFv-Fc type or diabodies, which generally have the same specificity of binding as the antibody from which they are descended. According to the present invention, antibody fragments of the invention can be obtained starting from antibodies such as described above by methods such as digestion by enzymes, such as pepsin or papain and/or by cleavage of the disulfide bridges by chemical reduction. In another manner, the antibody fragments comprised in the present invention can be obtained by techniques of genetic recombination likewise well known to the person skilled in the art or else by peptide synthesis by means of, for example, automatic peptide synthesizers such as those supplied by the company Applied Biosystems, etc.

In a more preferred manner, the invention comprises the antibodies, or their functional fragments, according to the present invention obtained by genetic recombination or by chemical synthesis.

In a preferred manner, said functional fragments according to the present invention will be chosen from the fragments Fv, scFv, Fab, (Fab′)₂, Fab′, scFv-Fc or diabodies, or any functional fragment whose half-life would have been increased by a chemical modification, especially by PEGylation, or by incorporation in a liposome.

The present invention also relates to an isolated nucleic acid comprising a sequence having at least 80%, preferably 85%, 90%, 95% and 98%, identity after optimum alignment with the sequence SEQ ID No. 5.

The present invention also relates to an isolated nucleic acid comprising a sequence having at least 80%, preferably 85%, 90%, 95% and 98%, identity after optimum alignment with the sequence SEQ ID No. 10.

By nucleic sequences having a percentage of identity of at least 80%, preferably 85%, 90%, 95% and 98%, after optimum alignment with a preferred sequence, it is intended to indicate the nucleic sequences having, with respect to the reference nucleic sequence, certain modifications such as, in particular, a deletion, a truncation, an elongation, a chimeric fusion and/or a substitution, especially point substitution. It preferably concerns sequences in which the sequences code for the same amino acid sequences as the reference sequence, this being connected to the degeneracy of the genetic code, or complementary sequences which are capable of hybridizing specifically with the reference sequences, preferably under conditions of high stringency, especially such as defined below.

A hybridization under conditions of high stringency signifies that the temperature conditions and ionic strength conditions are chosen in such a way that they allow the maintenance of the hybridization between two fragments of complementary DNA. By way of illustration, conditions of high stringency of the hybridization step for the purposes of defining the polynucleotide fragments described above are advantageously the following.

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a 0.15 M NaCl+0.015 M sodium citrate solution), 50% of formamide, 7% of sodium dodecyl sulfate (SDS), 10×Denhardt's, 5% of dextran sulfate and 1% of salmon sperm DNA; (2) actual hybridization for 20 hours at a temperature dependent on the size of the probe (i.e.: 42° C., for a probe size >100 nucleotides) followed by 2 washes of 20 minutes at 20° C. in 2×SSC+2% of SDS, 1 wash of 20 minutes at 20° C. in 0.1×SSC+0.1% of SDS. The last wash is carried out in 0.1×SSC+0.1% of SDS for 30 minutes at 60° C. for a probe size >100 nucleotides. The hybridization conditions of high stringency described above for a polynucleotide of defined size can be adapted by the person skilled in the art for oligonucleotides of greater or smaller size, according to the teaching of Sambrook et al., (1989, Molecular cloning: a laboratory manual. 2nd Ed. Cold Spring Harbor).

The invention also relates to a vector comprising a nucleic acid as defined above, in particular a nucleic acid of SEQ ID No. 5 and SEQ ID No. 10.

The invention aims especially at cloning and/or expression vectors which contain a nucleotide sequence according to the invention. 9. For example, it is aimed at baculovirus transfer vector comprising the nucleic acid sequence as defined above, especially of SEQ ID No. 5 and SEQ ID No. 10.

The vectors according to the invention preferably contain elements which allow the expression and/or the secretion of the nucleotide sequences in a determined host cell. The vector must therefore contain a promoter, signals of initiation and termination of translation, as well as appropriate regions of regulation of transcription. It must be able to be maintained in a stable manner in the host cell and can optionally have particular signals which specify the secretion of the translated protein. These different elements are chosen and optimized by the person skilled in the art as a function of the host cell used. To this effect, the nucleotide sequences according to the invention can be inserted into autonomous replication vectors in the chosen host, or be integrative vectors of the chosen host.

Such vectors are prepared by methods currently used by the person skilled in the art, and the resulting clones can be introduced into an appropriate host by standard methods, such as lipofection, electroporation, thermal shock, or chemical methods.

The vectors according to the invention are, for example, vectors of plasmidic or viral origin. They are useful for transforming host cells in order to clone or to express the nucleotide sequences according to the invention.

The invention likewise comprises the host cells transformed by or comprising a vector according to the invention.

The host cell can be chosen from prokaryotic or eukaryotic systems, for example bacterial cells but likewise yeast cells or animal cells, in particular mammalian cells. It is likewise possible to use insect cells or plant cells.

Thus, according to another aspect, the invention relates to a cell line secreting the above defined anti-R7V human antibody. For example, the above antibody may be obtained by EBV immortalized B lymphocytes, insect cells such as Sf9 cells using a baculovirus vector; or other antibody producing cell lines such as CHO (ATCC number CCL-61), genetically modified CHO to produce low fucosylated antibodies, or YB2/0 (ATCC CRL-1662) cell lines.

According to another aspect, the invention is aimed at a method of production of an antibody, or one of its functional fragments according to the invention, comprising the steps of:
a) culturing in a medium and appropriate culture conditions of a host cell according to the invention; and
b) extracting said antibodies from the culture medium of said cultured cells.

The antibodies, or one of their functional fragments, capable of being obtained by the above method are within the scope of the invention.

According to still another aspect, the invention relates to an antibody as defined above, or one of its functional fragments, as a medicament. It also concerns a pharmaceutical composition comprising as active principle an antibody, or one of its functional fragments according to the invention, and an excipient and/or a pharmaceutically acceptable vehicle.

In still another embodiment, the invention is directed to a composition such as described above which further comprises as a combination product for simultaneous, separate or sequential use, at least one agent currently used in therapy of AIDS and antibody according to the above. “Simultaneous use” is understood as meaning the administration of the two compounds of the composition according to the invention in a single and identical pharmaceutical form. “Separate use” is understood as meaning the administration, at the same time, of the two compounds of the composition according to the invention in distinct pharmaceutical forms. “Sequential use” is understood as meaning the successive administration of the two compounds of the composition according to the invention, each in a distinct pharmaceutical form. For example, it is possible to combine the administration of the anti-R7V antibody with:

efavirenz+zidovudine+lamivudine
efavirenz+tenofovir+emtricitabine
stavudine+lamivudine+nevirapine
lopinavir boosted with ritonavir+zidovudine+lamivudine
lopinavir boosted with ritonavir+tenofovir+emtricitabine.

The present invention comprises the use of the antibody depicted herein for the preparation of a medicament, especially for treating HIV infection, AIDS, for example in patients under HAART treatment and in particular in patients in failure of HAART treatment.

The invention will be further illustrated in view of the following figures and examples.

### LEGEND OF THE FIGURES

FIG. 1: Schematic representation of immunoglobulin specific transfer vectors used for the expression of anti-R7V antibody.

FIG. 1A: Schematic representation of pVT-Ck—Transfer vector allowing expression of the light chain.

FIG. 1B: Schematic representation of pVT-Cγ1—Transfer vector allowing expression of the heavy chain.

FIG. 2: PCR amplification of VH (FIG. 2A) or VL (FIG. 2B) sequences present on c-DNAs synthesized from total RNA extracted from immortalized B-lymphocytes selected on R7V antigen. The amplification was performed as reported in Materials and Methods with appropriate constant 3′ primer and sets of 5′ primers specific of a given VH or VL gene family. Twenty μl of PCR reaction were fractionated on a 1.5% agarose gel and stained with ethidium bromide. Lane C_{VH}: control VH sequence. Lane C_{VL}: control VL sequence. Lane MW: SmartLadder molecular weight marker (Eurogentec): 200, 400, 600, 800, 1000, 1500, 2000, 2500, 3000, 4000, 5000, 6000, 8000, 10,000 bp.

FIG. 3: FIG. 3A and FIG. 3C: Nucleotide sequences and FIG. 3B and FIG. 3D: amino-acid sequences of variable region of light (K4) and heavy (M4) chain of the antibody expressed in immortalized B-lymphocytes compared to the most homologous germline gene. Amino acid sequence are given in the one letter code. The numbering system used is based on the convention of IMGT (http://imgt.cines.fr). The complementary determining regions (CDR) of VH and VL sequences are highlighted. Dashes in sequences indicate identity with the residues given in the top line. IGHJ, IGHD and IGKJ genes are boxed.

FIG. 4: Neutralization of HIV₁ clades by 50 μg/ml of anti-R7V or irrelevant antibodies.

### EXAMPLES

### Example 1

### Isolation and Construction of an Effective Human Recombinant Anti-R7V Antibody

### 1.1 Material and Methods

### 1.1.1 Cells and Viruses

Human peripheral blood mononuclear cells (PBMC) were separated from fresh K2E-EDTA blood samples from healthy seronegative donors by Ficoll-Paque (Amersham) gradient centrifugation. Cultured cells were grown at a density of 1×10⁶ cells/ml in complete RPMI medium with the following composition: RPMI 1640 (Biowhittaker) supplemented with 10% heat-inactivated fetal calf serum (GIBCO), 1% penicillin/glutamine (GIBCO), 10 UI/ml IL2 (Euromedex), 10 μg/ml PHA-P (Difco) during the first 3 days, and 2 μg/ml polybrene (Biowhittaker).

CEM cell line was cultured at 0.5×10⁶ cells/ml in RPMI-10% culture medium (RPMI 1640 containing 10% heat-inactivated fetal calf serum, 1% penicillin/glutamine, 2 μg/ml polybrene).

The NDK (Glade D) and AZT-resistant RTMC (Glade B) viruses were produced on infected CEM cells. The 92UG029 (Glade A), 92BR021 (Glade B), 92BR025 (Glade C), and 93BR029 (Glade F) viruses were initially provided by the AIDS Research and Reference Reagent Program, Division of AIDS, NIAID, NIH and produced on PBMC. The viruses BCF06 (Glade 0), and YBF30 (old Glade) were kindly provided by F. Barre-Sinoussi (Pasteur Institute, France). Titrated viral aliquots from infected cells supernatants were kept frozen at −80° C.

Sf9 cells were maintained at 28° C. in TC100 medium (GIBCO) supplemented with 5% heat-inactivated fetal calf serum (GIBCO). Wild-type Autographa californica multiple nuclear polyhedrosis (AcMNPV) virus clone 1.2¹⁷ and recombinant baculoviruses were propagated in Sf9 cells.

1.1.2 Isolation of Peripheral Blood Mononuclear Cells (PBMC) from Non-Progressor Patients

Informed and consenting HIV seropositive non-progressor patients were enrolled in our study and the PBMC were purified from fresh K2E-EDTA venous blood by isolation on Ficoll-Paque density gradient. These PBMC were pre-cultivated 2 days in RPMI 1640 culture medium supplemented with 15% heat-inactivated FCS and 1% penicillin/glutamine without IL2 and PHA to favour the growth of B lymphocytes before immortalization.

1.1.3 Immortalization of B Lymphocytes with Epstein-Barr Virus (EBV)

B Lymphocytes were then immortalized by mixing 2 ml of B-95.8 culture supernatant (EBV producing cell line) with 9×10⁶ pre-cultivated PBMC in 3 ml 10% heat-inactivated FCS, 1% penicillin/glutamine RPMI 1640 in a 50 ml conical tube. After 2 hours incubation in a 37° C. water bath, 5 ml of RPMI 1640 supplemented with 10% heat-inactivated FCS, 1 μg/μl cyclosporin A (Calbiochem) and 1% penicillin/glutamine were added. The 10-ml cell suspension were transferred to a 25 cm² tissue-culture flask in a humidified 37° C., 5% CO₂ incubator and cultured undisturbed for 4 weeks. At the end of the 4-week incubation, the EBV-immortalized cells formed macroscopic clumps and this cell line was maintained by re-feeding twice a week at 10⁶ cells/ml in RPMI-20%.

### 1.1.4 Separation of B Lymphocytes Secreting Anti-R7V Antibodies

Coating of magnetic beads by an aminohexanoic acid form of R7V peptide: 10 μg of R-8-Ahx peptide (Neosystem) were incubated with 10⁷ magnetic tosyl-activated beads (Dynal Dynabeads® M450) 16-24 hours at 37° C. with slow tilt rotation. Beads were washed according the manufacturer procedures and resuspended at 4.10⁸ beads/ml in a PBS pH 7.4.

Magnetic selection of anti-R7V antibodies secreting B lymphocytes: 10⁷ EBV-immortalized B lymphocytes in 1 ml sterile PBS are mixed with 24 10⁶ R-8-Ahx coated beads during 20 min at 4° C. and repeated three times until no more cell fixed the beads. The rosetted cells were isolated by placing the tube in a magnet for 2 minutes. The supernatant was removed without disturbing the beads, and the cells were resuspended in a PBS washing buffer. The washing step was repeated 3 times before cultivating beads-fixed cells in RPMI-20% FCS at 37° C., 5% CO₂. One day after, the cells detached themselves from the beads and grew at 10⁶ cells/ml.

This magnetic selection was repeated after two weeks of culture with the same protocol on these pre-selected anti-R7V antibodies secreting B lymphocytes.

### 1.1.5 ELISA Procedure

Anti-R7V antibodies were detected by an anti-R7V ELISA assay (Anti R7V™ IVR96000, IVAGEN, France) as indicated by the manufacturer. Briefly, positive, negative controls, a cut-off calibrator and diluted antibodies (100 μl/well) were added to a R7V-coated test plate and incubated 30 min at room temperature. Bound anti-R7V antibodies were detected by an horseradish peroxidase-conjugated anti-human IgG antibody.

### 1.1.6 Neutralization Assay

Viral stocks were titrated previously to have 100 TCID50 per assay¹⁸ corresponding to the following dilutions: HIV-1_{NDK} (dilution 10⁻⁵), HIV-1_{RTMC} AZT-resistant (dilution 5 10⁻⁵), 92UG029 (dilution 10⁻²), 92BR021 (dilution 10⁻³), 92BR025 (dilution 10⁻²), THA92022 (dilution 10⁻²), 93BR029 (dilution 10⁻²), BCF06 (dilution 10⁻⁴) and HIV-1_{YBF30} (dilution 10⁻³). Dilution of viruses (50 μl) were pre-incubated in 96-well microtiter plate in 50 μl RPMI-0% containing 100 μg/ml of antibody (final concentration 50 μg/ml) during 1 h in a humidified 37° C., 5% CO₂ incubator. PBMC (1×10⁶ in 50 μl) were added to the virus-antibody mixture for 1 h at 37° C. and cells were washed three times with culture medium and cultured at 10⁶ cells/ml in 24-well microtiter plate in presence of 50 μg/ml antibody complete RPMI-10% during the first 3 days. Cultures were grown for 10 days and re-fed every 3 days. The same assays were done for virus control (HIV-infected cells without antibody), cells control (uninfected cells without antibody) and antibody control (irrelevant antibody directed against a non HIV-related epitope. To measure the viral replication in each sample, the reverse trancriptase enzyme was quantified as follow. One milliliter samples of cell-free supernatant collected every three days were ultracentrifuged at 95,000 rpm, 4° C., 5 min (TL100 Beckman). The viral pellet was resuspended in 10 μl of 0.1% Triton X-100 NTE (NaCl 100 mM, Tris 10 mM, EDTA 1 mM) buffer to release viral enzymes. The enzymatic reaction was performed in 50 μA of a reaction mixture containing Tris 50 mM, pH 7.8; MgCl₂ 20 mM; KCl 20 mM; dithiothreitol (DTT) 2 mM; oligo dT 0.25 OD/ml; poly rA 0.25 OD/ml and³H dTTP 50 μCi/ml. After 1 h at 37° C., the reaction was stopped with 1 ml sodium pyrophosphate in 5% TCA and the synthetized DNA products were precipitated with 20% trichloroacetic acid, collected by filtration on Millipore 0.45 μm and the 13 radioactivity was measured in dpm/ml on a Packard scintillation counter. Percentages of neutralization were expressed as:

[100−(Reverse transcriptase activity of the sample/Reverse transcriptase activity of the virus)×100)].

### 1.1.7 Isolation and Cloning of the Variable Regions of Antibodies Expressing the Anti R7V Specificity

The procedure was adapted from the technique described for the amplification of murine variable antibody regions¹⁹. Total RNA was extracted from about 5.10⁶ immortalized B lymphocytes using the RNeasy kit (Qiagen). Briefly, cells were lyzed with 600 μl of RLT™/β-mercaptoethanol buffer and homogenized by serial passages through 20 gauge needle. After addition of 600 μl of 70% ethanol, the mixture was deposited on RNeasy column and centrifuged for 15 s at 12,000 rpm (Biofuge, Heraeus). Column was washed successively with 700 μl RW1™ buffer and with 500 μl RPE™ buffer. RNAs were eluted with 50 μl RNAse-free water and conserved at −80° C. until use.

Total RNA and five specific primers hybridizing in the constant regions of human immunoglobulins, hCLa, hCLb, hCK, hCG and hCM (Table 3) were used to synthesize first strand c-DNAs corresponding to lambda, kappa, gamma 1 and mu mRNA respectively. Reverse-transcriptions were carried out as follows: 1 μg of total RNA, 4 μl of 10×RT™ buffer (Qiagen), 4 μl of 5 mM of each dNTP (Qiagen), 4 μl of the specific primer at 10 pMoles/μl 20 units of RNAse inhibitor (Roche) and 8 units of Omniscript reverse transcriptase (Qiagen) in a final volume of 40 μl. Mixtures were incubated for 1 hour at 37° C. Reverse transcription activity was heat-inactivated at 93° C. for 5 min. Full length VH and VL sequences were amplified by PCR using specific primers designed in the signal peptide sequence of heavy and light chains of human immunoglobulins (Table 3) and lambda, kappa, gamma or mu first-strand cDNA as a matrix. The PCR reactions were carried out in a final volume of 20 μA containing 2 μA of 10X Vent DNA polymerase (Biolabs), 2 μA of 10 mM each dNTP (Biolabs), 20 pMoles of each primers, 1.5 μA of 25 mM MgSO₄, 1 unit of Vent DNA polymerase (Biolabs), 0.5 μA of reverse transcription mixture. Thirty cycles of amplification were performed for 30 s at 95° C., 45 s at 55° C. and 1 min at 72° C. After a 10 min extension at 72° C., PCR products were fractionated on a 1.5% agarose gel (SeaKem, FMC) and stained with ethidium bromide.

PCR products were gel purified, amplified with Advantage Taq polymerase (Clonetech) and cloned in plasmid pGemT easy (Promega). Inserts were sequenced on both strands using the 3′ and 5′ primers used for the PCR amplification (MWG Biotech). Sequence comparison and germline gene analysis of variable regions were performed using BLAST²⁰ and IMGT Database²¹.

1.1.8 Construction of a recombinant baculovirus expressing anti-R7V antibodies

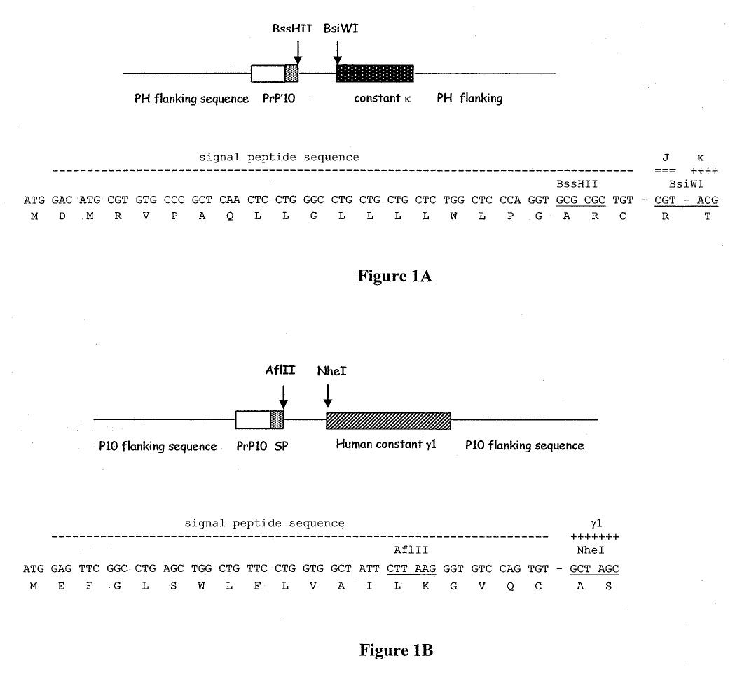

VH and VL sequences were inserted in specific transfer vectors pVTCγ1 and pVTCκ(FIG. 1) containing a human immunoglobulin signal peptide sequence, two unique restriction sites and sequences encoding human gamma 1 and kappa constant region respectively. The pVTCγ1 vector contains a unique AflII site in the signal peptide sequence and a NheI site comprising the two first codons of the gamma 1 sequence while pVTCκ contains a unique BssHII site in the signal peptide sequence and a BsiWI site overlapping the last conserved amino-acid of J region and the first amino-acid of the constant kappa region.

Appropriate restriction sites were introduced at the 5′ and 3′ ends of VH and VL sequences by PCR using the following primers:

FOR-M4:
(SEQ ID N^{o} 16)
CCATCTTAAGGGTGTCCAGTGTCAGGTGCAGCTGCAGGAGTCGGGCCCA
GGACTGGTGAAGC,
BAC-M4:
(SEQ ID N^{o} 17)
GCATGCTAGCTGAGGAGACGGTGACCAGGGT,
FOR-K4:
(SEQ ID N^{o} 18)
CGATGCGCGCTGTGACATCGTGATGACCCAGTCT
and
BAC-K4:
(SEQ ID N^{o} 19)
CGATCGTACGTTTGATCTCCAGCTTGGTCCCCTGGCC.

PCR products digested with AflII-NheI for VH and BssHII-BsiWI for VL were purified and inserted in their respective transfer vectors pVTCγ1 and pVTCκ. The final constructs pVTCγ1-M4 and pVTCκ-K₄ were controlled by sequencing. Recombinant baculoviruses expressing the antibody were generated after cotransfection of Sf9 cells as previously described (^{22, 10, 11}). Productive clones were screened by ELISA²³. Briefly, microtiter plates coated with 100 μA of 1 μg/ml of anti-human heavy chain Fdγ1 polyclonal antibody (The Binding Site) were incubated with serial dilutions of cell culture supernatants for 2 hours at 37° C. Bound recombinant IgG was detected using horseradish peroxidase-labeled anti-human kappa light chain antibody (Sigma). The genome of recombinant viruses was controlled by Southern blot. Viral particles in 7 ml of cell culture supernatant were sedimented at 35,000 rpm for 40 minutes (TL100.4, Beckman). Pellets were resuspended in 1 ml of TEK buffer (0.1 M Tris, 0.1 M Na₂EDTA 2 H₂O, 0.2 M KCl, pH 7.5) in the presence of 10 μA of proteinase K at 20 mg/ml in water (Roche) and 10 μA of N-lauryl sarcosine (Sigma) at 10% (w/v) in water and incubated at 50° C. overnight. Viral DNA was successively extracted with phenol and chloroform-isoamyl alcohol (24:1 v/v) and precipitated with ethanol. After resuspension in water, DNA was digested with HindIII. Restricted DNA was then analysed by electrophoresis on 1% agarose gel and transferred onto a Nitran membrane (Schleicher and Schull). The c-DNAs encoding human constant γ1 and constant κ region respectively were labelled with digoxigenin (Roche) as recommended by the manufacturer and used as hybridization probes. After washes, blots were incubated with antidigoxin antibodies conjugated to alkaline phosphatase (Roche, dilution 1:10,000). Detection of labelled DNA was carried out with the chemioluminescent substrate CSPD (Roche).

### 1.1.9 Production and Purification of Recombinant Antibodies

Sf9 cells were seeded at a density of 500,000 cells/ml in 400 ml of serum free medium in roller bottles and infected at a multiplicity of infection of 2 per cell. After 4 days incubation at 28° C., supernatant was collected and secreted recombinant antibodies were purified on protein A sepharose (Amersham) as indicated by the manufacturer. The quantity of purified IgG was measured by ELISA²³.

Recombinant anti-R7V antibodies were also constructed in CHO-expressing system under similar conditions.

### 1.2 Results

1.2.1 Selection of anti-R7V antibodies secreting B lymphocytes

Anti-R7V antibodies producing B lymphocytes were selected from a non-progressor HIV-infected patient using R7V-coated magnetic beads. Twenty-seven percent of B lymphocytes secreting anti-R7V antibodies were obtained at the first selection, and 14% at the second one done on the pre-selected anti-R7V antibodies secreting B lymphocytes. No free anti-R7V antibodies were detected by anti-R7V ELISA in the B cell culture supernatant, suggesting that antibodies were either bound to the secreting B lymphocytes membrane or below the limit of detection of the ELISA test.

### 1.2.2 Isolation and Cloning of VH and VL Sequences Expressed by the Selected Immortalized B-Lymphocytes.

Amplification of VL and VH regions of antibodies expressed by the selected B lymphocytes were performed by RT-PCR as we previously described for mouse immunoglobulins¹⁹.

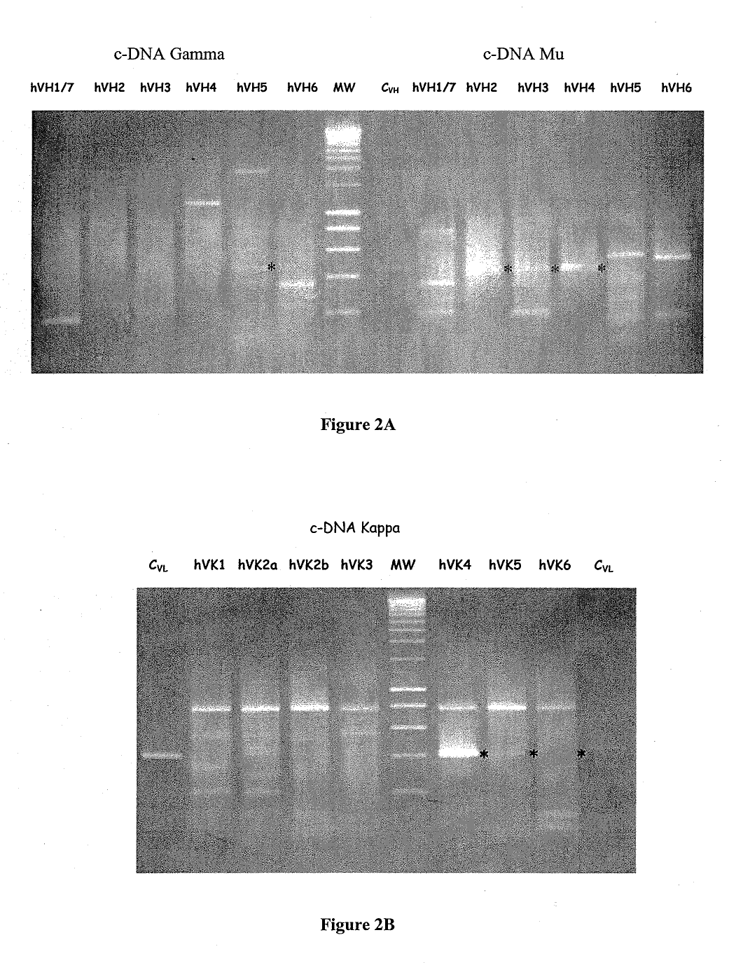

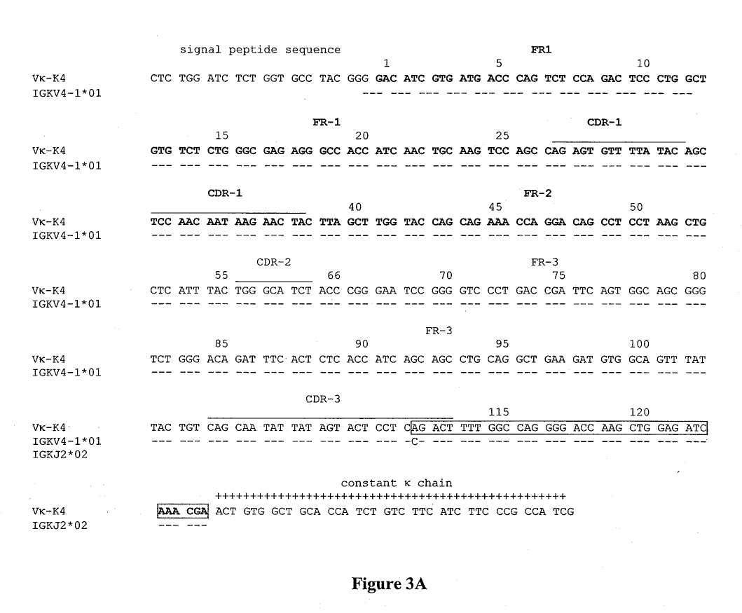

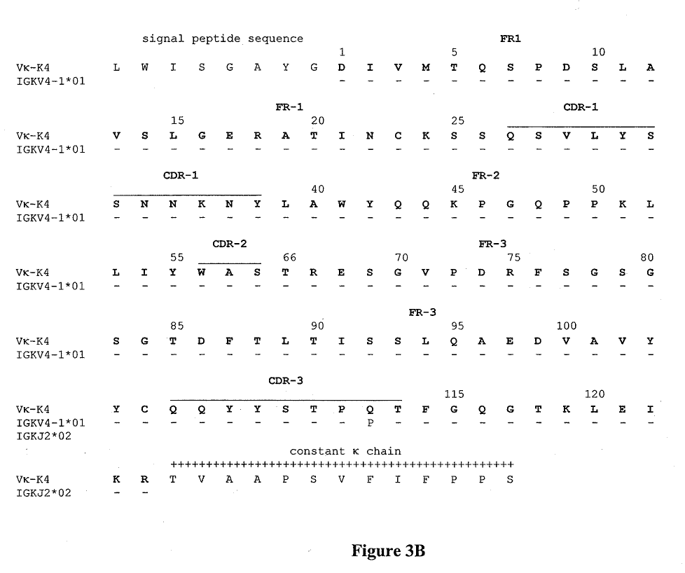

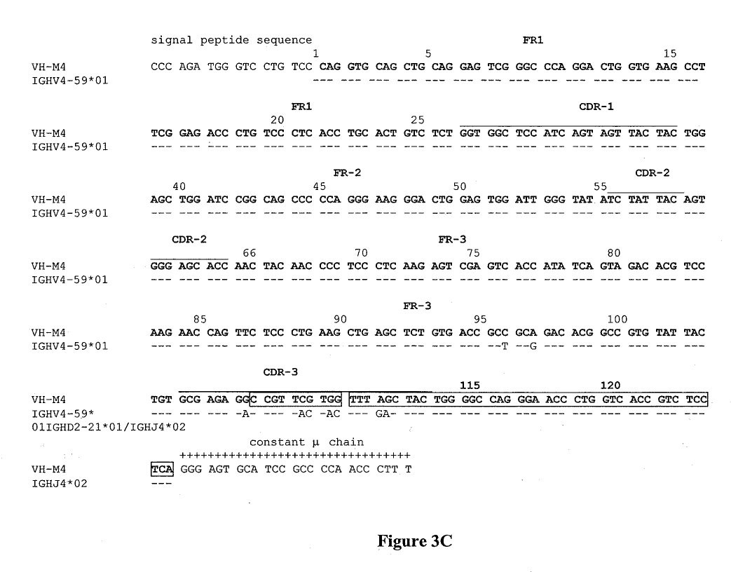

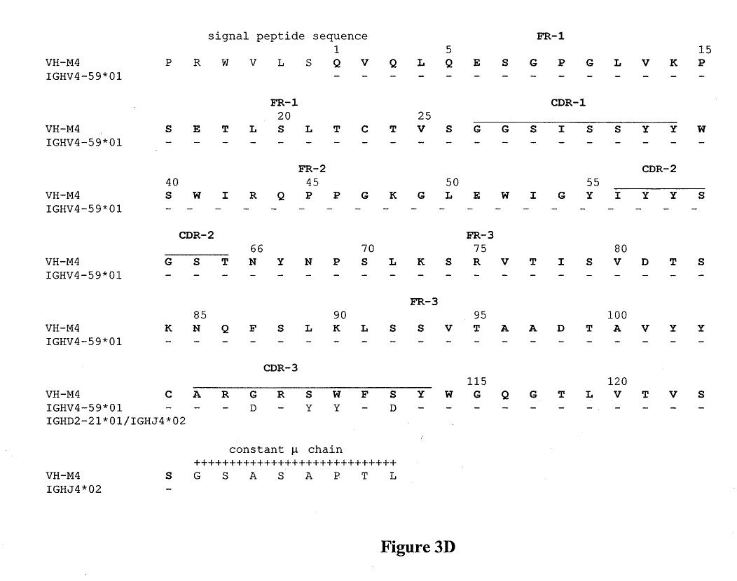

As shown on FIG. 2, only few combinations of primers led to the amplification of fragments with the appropriated size, about 450 bp for VH and 400 bp for VL. While only faint bands were observed using hCG/hVH5, hCM/hVH2 and hCM/hVH3, more material was observed with hCM/hVH4. A major product was also synthesized with hCK/hVK4. However, no amplification was detected with any combination using hCLa and hCLb primers (not shown). Sequencing and BLAST analysis of the PCR products showed that only two of them, M4 fragment (hCG/hVH4) and K₄ fragment (hCK/hVK4) corresponded to a variable domain of human heavy and light chain respectively. These results indicates that the population of selected immortalized B-lymphocytes is probably monoclonal, expressing a membrane IgM kappa antibody. Comparison of these sequences with IMGT database shows that VH-M4 heavy chain variable region sequence results from the rearrangement of IGHV-4-59*01²⁴, IGHD2-21*01²⁵ and IGHJ4*02²⁶ germline genes (FIG. 3C). Its Vκ-K4 counterpart shows a IGKV4-1*01²⁷/IGKJ2*02²⁸, rearrangement of the light chain variable region (FIG. 3A). Interestingly this antibody used the most J-proximal IGKV4-1 gene from the kappa light chain repertory. Such light chain region was mainly unmutated, with only one mutation in the complementary determining region 3 at the IGKV/IGKJ junction, (FIG. 3B). On the other hand, seven nucleotide replacements leading to four amino-acid mutations in the complementary determining region 3 were observed in the VH-M4 sequence whereas only two silent nucleotide replacements were noted in the framework regions (FIGS. 3C, 3D).

### 1.2.3 Expression of the Anti-R7V Antibody in the Baculovirus Expression System

The sequences encoding the variable regions of the anti-R7V antibody were inserted in the light and heavy chain cassette baculovirus transfer vectors (i) pVT-CK designed to recombine in the polyhedrin locus and (ii) pVT-Cγ1 designed to recombine in the P10 locus. In these constructs, the light and heavy chains genes are under the control of a synthetic P10 promoter, P′10²² and the P10 promoter respectively (FIG. 1). Specific primers were designed to amplify K4 and M4 fragments allowing their direct cloning in frame with the immunoglobulin signal peptide sequence and the constant region as shown on FIG. 1. The two final constructs, pVT-Ck-K4 and pVT-Cγ1-M4 were controlled by sequencing and used to cotransfect Sf9 cells in the presence of purified viral DNA. Double recombinant viruses were obtained after two rounds of recombination as described previously^{10,11}. Recombinant viruses were plaque purified and amplified. The presence of antibody in the cell culture supernatant of infected cells was analyzed by an anti-human antibodies ELISA. The genomes of four productive clones were controlled by southern blotting using human γ1 and k constant regions DNAs as probes. One viral clone named AcR7VI/K4-M4 was selected for further experiments.

### 1.2.4 Specificity of the Recombinant Anti-R7V Antibody

Recombinant anti-R7V antibodies were positive in the IVAGEN Anti-R7V ELISA kit, even at 6.25 μg/ml corresponding to a concentration of 0.625 μg of antibodies in the well. Irrelevant antibodies were negative whatever their concentration.

As previously reported for anti-R7V antibodies purified from non-progressor patients, the recombinant monoclonal antibody doesn't bind to any cell as demonstrated by flow cytometry analysis (data not shown).

### 1.2.5 Neutralization Assay for Several Clades of HIV-1

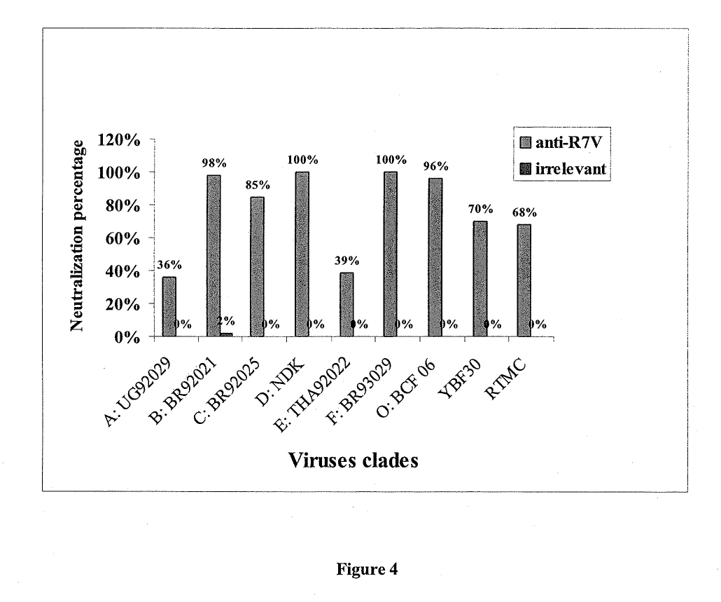

The anti-R7V antibodies purified from patients were described to display a broad neutralizing spectrum, so this anti-R7V monoclonal antibody was tested under the same conditions against several clades. To ascertain its use as therapeutic antibody, the neutralization assay was also done with a drug-resistant virus (RTMC). To measure the neutralizing effect of the anti-R7V recombinant antibodies, a 50 μg/ml dilution of antibody was mixed with several clades of HIV-1 before infecting the cells. The anti-R7V antibody neutralized 8 clades of HIV-1 and the AZT-resistant Glade B RTMC virus (FIG. 4). No neutralization was observed for the irrelevant antibodies expressed in baculovirus system and used as control under the same conditions. More than 85% neutralization were obtained for 5 clades (B, C, D, F, and O). Different percentages of neutralization were obtained with 50 μg/ml of recombinant anti-R7V antibody according to the different viruses. This heterogeneous results, identical to those of purified anti-R7V antibodies from HIV non-progressor patients, are probably due to the variable amount of R7V epitope presented by the viruses.

Construction of recombinant anti-R7V antibodies in CHO-expressing system

1) K4M4 Lot Number 13.11.06: Anti-R7V ELISA Result Positif at 40 μg/ml

TABLE 1
Neutralisation percentages by anti-R7V antibodies

| | | Anti-R7V antibody concentration (μg/ml) | | |
|---|---|---|---|---|
| virus | clade | 70 μg/ml | 50 μg/ml | 25 μg/ml |
| RW92009 | A | | 30% | 12% |
| YBF30 | N | 25% | 10% | 21% |
| BCF06 | O | 30% | 48% | |
| BR92021 | B | 24% | 12% | |
| BR92025 | C | 77% | 47% | |
| BR93029 | F | 18% | 11% | |

2) K4M4 Lot Number 28.02.07: Anti-R7V ELISA Result Positif at 50 μg/ml

TABLE 2
Neutralisation percentages by anti-R7V antibodies

| | | Anti-R7V antibody concentration (μg/ml) | | | |
|---|---|---|---|---|---|
| virus | clade | 70 μg/ml | 50 μg/ml | 25 μg/ml | 10 μg/ml |
| RW92009 | A | | 60% | 12% | |
| BR92021 | B | | 30% | | |
| UG92035 | D | | 74% | 26% | 51% |

### 1.3 Conclusion

We have reported here the results concerning the production of a recombinant human anti-R7V antibody by the baculovirus expression system. This system is very fast and efficient for the production of large amount of functional recombinant antibody^{10, 11, 29} All post translational modifications observed in mammalian cells are found on recombinant proteins expressed in lepidopteran cells. However, N-linked oligosaccharides are shorter and essentially of high mannose or paucimannose types^{30,31}. Biological activity of antibodies is highly dependent of the N-glycans linked to the Asn-292 in the CH₂ constant domain of immunoglobulin^{32,33}. Despite this uncomplete glycosylation pattern, recombinant antibodies expressed in Sf9 cells exhibit specific biological activities such as complement dependent and antibody-dependent cell-mediated cytotoxicity through Clq and Fc7R binding^{14, 16, 13}.

In order to isolate and characterize anti-R7V antibodies produced by HIV-1 non-progressor patients, EBV-immortalized R7V-reactive B cells were selected from one patient and the c-DNAs encoding the variable regions of IgG or IgM immunoglobulins were specifically amplified using RT-PCR. For this purpose, three original sets of consensus primers were designed for the specific amplification of the human VH and VL regions whatever the V gene family.

These primers hybridizing in the signal sequence were used in conjunction with a set of 3′ primers directed to the human constant regions γ, μ, κ and λ respectively. In contrast to the framework 1 domain targeted in “FR” amplification strategies which can undergo somatic mutations^{34,35}, the frequency of mutation in signal sequence is very low, so, priming in this region, allows the amplification of entire sequence without mutations.

Analysis of the sequence of these c-DNAs showed that these immortalized cells are probably monoclonal expressing only one membrane IgM kappa antibody. While VL sequence is largely unmutated with only one silent mutation at the VJ junction, 6 mutated amino acids are found in the CDR3 at the VDJ junction of VH domain with only 2 silent mutations in the FR3. Despite the low mutation rate observed in its variable regions, this recombinant antibody is not polyreactive as it does not react with any cells following flow cytometry analysis.

The neutralization capacity of this fully human recombinant antibody on HIV-1 subtypes A, B, C, D, E, F, N, O and on an antiretroviral therapy-resistant virus is clearly identical to polyclonal antibodies from non-progressor patients. These results confirm the acquisition, by all HIV-1 variants, of the cellular-derived R7V epitope. The different percentages of neutralization obtained with 50 μg/ml of anti-R7V antibody are probably be linked to the various amount of R7V present on the viruses. Increasing amounts of antibody have to be tested to reach 100% of neutralisation for each Glade.

One of the most important qualities for a monoclonal antibody, to act as a therapeutic agent for HIV-infected patients, is its broad spectrum of neutralization. It's known that HIV changes continuously from an individual to another according to the time of infection and to the antiretroviral treatment (apparition of escape mutants), explaining the difficulties for the immune system to control the viral replication. Today, apart from anti-R7V antibodies, four other broadly neutralizing monoclonal antibodies, all raised against HIV-1 subtype B, show such potency. IgG1b12, directed against the CD4 binding site on the surface gp120, has been generated from an asymptomatic HIV-positive individual by the phage display technique^{36,37,38}. The 2F5 and 4E10 antibodies recognize a constant part of the gp41^{39,40}, whereas 2G12 is raised against an epitope on the gp120^{41,42}. These four antibodies are reported as broadly neutralizing antibodies, but the most effective effect was obtained when they were mixed together^{43,44}.

In our results, we have shown that the recombinant anti-R7V antibody neutralizes the HIV-1 subtype C isolate. On this subtype, monoclonal antibodies 2F5 and 2G12 are ineffective, IgG1b12 partially effective and only 4E10 shows a significant activity⁴⁵.

So, the anti-R7V antibody appears to be one of the most broadly effective Mab against HIV-1 described to date. Despite its cellular origin, the R7V epitope is not responsible of autoimmune responses, as none of the patients producing anti-R7V antibodies has any clinical sign of autoimmune disease⁵. This confirms that this anti-R7V antibody is a powerful candidate for a therapy of HIV-infected patients.

TABLE 3
Gene family-specific PCR primers used to screen human lymphocytes c-DNA
for identification of light and heavy chain variable region

| | Sequence of primers | Sequence of primers |
|---|---|---|
| | hybridizing in the signal | hybridizing in the |
| | peptide sequence of human Ig | constant region of human Ig |
| Heavy | hVH1/VH7 ATGGACTGGACCTGGAG | Gamma |
| chain | (SEQ ID N^{o} 20) | hCG GGAAGTAGTCCTTGACCAGGCAG |
| | hVH2 ATGGACATACTTTGTTCC (SEQ ID N^{o} 21) | (SEQ ID N^{o} 26) |
| | hVH3 ATGGAGTTTGGGCTGAGC (SEQ ID N^{o} 22) | Mu |
| | hVH4 ATGAAACACCTGTGGTT (SEQ ID N^{o} 23) | hCM GGAGACGAGGGGGAAAAGGGT |
| | hVHS ATGGGGTCAACCGCCATC (SEQ ID N^{o} 24) | (SEQ ID N^{o} 27) |
| | hVH6 ATGTCTGTCTCCTTCCTC (SEQ ID N^{o} 25) | |
| Light | Lambda | Lambda |
| chain | hVL1a TCACTGCACAGGSTCCWGGGCC | hCLa CTCAGAGGAGGGCGGGAACAGAGTGAC |
| | (SEQ ID N^{o} 28) | (SEQ ID N^{o} 42) |
| | hVL1b TCACTGTGCAGGGTCCTGGGCC | hCLb CTCAGAGGACGGCAGGAACAGAGTGAC |
| | (SEQ ID N^{o} 29) | (SEQ ID N^{o} 43) |
| | hVL2 CTCCTCACTCAGGRCACAGG (SEQ ID N^{o} 30) | Kappa |
| | hVL3a CTCCTCACTYTCTGCACAG (SEQ ID N^{o} 31) | hCK GATGGCGGGAAGATGAAGACAGATGG |
| | hVL3b CTCCTCTCTCACTGCACAG (SEQ ID N^{o} 32) | (SEQ ID N^{o} 51) |
| | hVL3c TCCTTGCTTACTGCACAGGA (SEQ ID N^{o} 33) | |
| | hVL3d TCACTCTTTGCATAGGTTCTGTG | |
| | (SEQ ID N^{o} 34) | |
| | hVL4 CTCCTCCTCCACTGSACAGGG (SEQ ID N^{o} 35) | |
| | hVL5 TTCCTCTCTCACTGCACAGG (SEQ ID N^{o} 36) | |
| | hVL6 CTCCTCGCTCACTGCACAG (SEQ ID N^{o} 37) | |
| | hVL7 CTCCTCACTYGCTGCCCAGGG (SEQ ID N^{o} 38) | |
| | hVL8 CTCCTTGSTTATGGRTCAGG (SEQ ID N^{o} 39) | |
| | hVL9 CTCCTCAGTCTCCTCACAGGG (SEQ ID N^{o} 40) | |
| | hVL10 CTCCTCACTCACTCTGC (SEQ ID N^{o} 41) | |
| | Kappa | |
| | hVK1 TCAGCTCCTGGGGCTYCTG (SEQ ID N^{o} 44) | |
| | hVK2a CTGGGGCTGCTAATGCTCTGG (SEQ ID N^{o} 45) | |
| | hVK2b CTGGGGCTGCTCCTGGTCTGG (SEQ ID N^{o} 46) | |
| | hVK3 TCCTGCTACTCTGGCTCCCAG (SEQ ID N^{o} 47) | |
| | hVK4 TGCTCTGGATCTCTGGTGC (SEQ ID N^{o} 48) | |
| | hVK5 CTCCTCCTTTGGATCTCTGATACCAGGGCA | |
| | (SEQ ID N^{o} 49) | |
| | hVK6 CTCTGGGTTCCAGCCTCCAGGGGT | |
| | (SEQ ID N^{o} 50) | |

Standard abbreviations are used for mixed sites: R = A or G, Y = T or C, W = A or T.

### REFERENCES

1. Oelrichs R, Tsykin A, Rhodes D, Solomon A, Ellett A, McPhee D, et al.: Genomic sequences of HIV type 1 from four members of the Sydney Blood Bank Cohort of long-term nonprogressors. AIDS Res Hum Retrovir 1998; 14:811-814.
2. Sanchez G, Xu X, Chemann J C and Hirsch I: Accumulation of defective viral genomes in peripherical blood mononuclear cells of Human Immunodeficiency Virus type 1-infected individual. J Virol 1997; 71:2233-2240.
3. Connor R I, Sheridan K E, Ceradini D, Choe S, Landau N R: Change in coreceptor use correlates with disease progression in HIV1-infected individuals. J Exp Med 1997; 185: 621-628.
4. De Roda Husman A M, Schuitemaker H: Chemokine receptors and the clinical course of HIV-1 infection. Trends Microbiol 1998; 6:244-249.
5. Haslin C, Chemann J C: Anti-R7V antibodies as therapeutics for HIV-infected patients in failure of HAART. Curr Opin Biotechnol 2002; 13(6):621-624.
6. Galea P, Le Contel C, Coutton C, Chemann J C: Rationale for a vaccine using cellular-derived epitope presented by HIV isolates. Vaccine 1999; 17:1700-1705.
7. Arthur L O, Béss J W Jr, Sowder R C 2nd, Benveniste R E, Mann D L, Chemann J C, et al.: Cellular proteins bound to immunodeficiency viruses: implications for pathogenesis and vaccines. Science 1992; 258:1935-1938.
8. Le Contel C, Galea P, Silvy F, Hirsch I, Chemann J C: Identification of the beta-2-microglobulin-derivated epitope responsible for neutralisation of HIV isolates. Cell Pharmacol 1996; 3:68-73.
9. Hasemann C A, Capra D: High-level production of a functional immunoglobulin heterodimer in a baculovirus expression system. Proc Natl Acad Sci USA 1990; 87: 3942-3946.
10. Poul M A, Cérutti M, Chaabihi H, Ticchioni M, Deramoult F X, Bernard A, et al.: Cassette baculovirus vectors for the production of chimeric, humanized, or human antibodies in insect cells. Eur J Immunol 1995; 25:2005-2009.
11. Poul M A, Cérutti M, Chaabihi H, Devauchelle G, Kaczoreck M, Lefranc M P: Design of cassette baculovirus vectors for the production of therapeutic antibodies in insect cells. Immunotechnology 1995; 1:189-196.
12. zu Putlitz J, Kubasek W L, Duchene M, Marget M, von Specht B U, Domdey H: Antibody production in Baculovirus-infected insect cells. Biotechnology (New York) 1990; 8:651-654.
13. Troadec S, Chentouf M, Cérutti M, Nguyen B, Olive D, Bés C, Chardes T: In vitro anti-tumoral activity of baculovirus-expressed chimeric recombinant anti-CD4 antibody 13B8.2 on T-cell lymphomas. J Immunother (in press) 2006.
14. Carayannopoulos L, Max E E, Capra J D: Recombinant human IgA expressed in insect cells. Proc Natl Acad Sci USA 1994; 91:8348-8352.
15. Nesbit M, Fu Z F, McDonald-Smith J, Steplewski Z, Curtis PJ: Production of a functional monoclonal antibody recognizing human colorectal carcinoma cells from a baculovirus expression system. J Immunol Methods 1992; 151: 201-208.
16. Edelman L, Margaritte C, Chaabihi H, Monchatre E, Blanchard D, Cardona A, et al.: Obtaining a functional recombinant anti-rhesus (D) antibody using the baculovirus-insect cell expression system. Immunology 1997; 91:13-19.
17. Croizier G, Croizier L, Quiot J-M, Lereclus D: Recombination of Autographa californica and Rachiplusia ou nuclear polyhedrosis viruses in Galleria mellonella L. J Gen Virol 1988; 69: 177-185.
18. Rey F, Donker G, Hirsch I, and Chemann J C: Productive infection of CD4+ cells by selected HIV strains is not inhibited by anti-CD4 monoclonal antibodies. Virology 1991; 181:165-171.
19. Chardes T, Villard S, Ferrieres G, Piechaczyk M, Cérutti M, Devauchelle G, et al.: Efficient amplification and direct sequencing of mouse variable regions from any immunoglobulin gene family. FEBS Lett 1999; 452:386-394.
20. Altschul S F, Madden T L, Sch [symbol] ffer A A, Zhang J, Zhang Z, Miller W, et al.: Gapped BLAST and PSI-BLAST: A new generation of protein database search programs. Nucleic Acids Res 1997; 25:3389-3402.
21. Lefranc M P, Giudicelli V, Kaasl Q, Duprat E, Jabado-Michaloud J, Scaviner D: IMGT, the international ImMunoGeneTics Information System®. Nucleic Acids Research 2005; 33:D593-D597.
22. Cérutti M, Chaabihi H, Devauchelle G, Gautier L, Kaczorek M, Lefranc M P, et al.: Recombinant Baculovirus and use thereof in the production of monoclonal antibodies. INRA-CNRS Patent, France, FR 94/01015 1994.
23. Bés C, Briant-Longuet L, Cérutti M, Heitz F, Troadec S, Pugniére M, et al.: Mapping the paratope of the anti-CD4 recombinant Fab 13B8.2 by combining parallel peptide synthesis and site-directed mutagenesis. J Biol Chem 2003; 278:14265-14273.
24. Van der Maarel S, van Dijk K W, Alexander C M, Sasso E H, Bull A, Milner E C: Chromosomal organization of the human VH4 gene family. Location of individual gene segments. J Immunol 1993; 150:2858-2868.
25. Siebenlist U, Ravetch J V, Korsmeyer S, Waldmann T, Leder P: Human immunoglobulin D segments encoded in tandem multigenic families. Nature 1981; 294:631-635.
26. Mattila P S, Schugk J, Wu H and Makela O: Extensive allelic sequence variation in the J region of the human immunoglobulin heavy chain gene locus. Eur J Immunol 1995; 25(9):2578-2582.
27. Klobeck H G, Bornkamm G W, Combriato G, Mocikat R, Pohlenz H D, Zachau H G: Subgroup IV of human immunoglobulin K light chains is encoded by a single germline gene. Nucleic Acids Res 1985; 13:6515-6529.
28. Sahota S S, Leo R, Hamblin T J, Stevenson F K: Myeloma VL gene sequences reveal somatic hypermutation with intraclonal homogeneity, and a role for VL in antigen selection (Unpublished, access number: Z70260) 1996.
29. Lieby P, Soley A, Levallois H, Hugel B, Freyssinet J M, Cérutti M: The clonal analysis of anticardiolipin antibodies in a single patient with primary antiphospholipid syndrome reveals an extreme antibody heterogeneity. Blood 2001; 97:3820-3828.
30. Marchal I, Mir A M, Kmiecik D, Verbert A, Cacan R: Use of inhibitors to characterize intermediates in the processing of N-glycans synthesized by insect cells: a metabolic study with Sf9 cell line. Glycobiology 1999; 9: 645-654.
31. Marchal I, Jarvis D L, Cacan R, Verbert A: Glycoproteins from insect cells: sialylated or not ? Biol Chem 2001; 382:151-159.
32. Boyd P N, Lines A C, Patel A K: The effect of the removal of sialic acid, galactose and total carbohydrate on the functional activity of Campath-1H. Mol Immunol 1995; 32:1311-1318.
33. Shinkawa T, Nakamura K, Yamane N, Shoji-Hosaka E, Kanda Y, Sakurada M, et al.: The absence of fucose but not the presence of galactose or bisecting N-acetylglucosamine of human IgG1 complex-type oligosaccharides shows the critical role of enhancing antibody-dependent cellular cytotoxicity. J Biol Chem 2003; 278:3466-3473.
34. Kettleborough C A, Saldanha J, Ansell K H, Bendig M M: Optimization of primers for cloning libraries of mouse immunoglobulin genes using the polymerase chain reaction. Eur J Immunol 1993; 23:206-211.
35. Orlandi R, Gussow, D H, Jones P T, Winter G: Cloning immunoglobulin variable domains for expression by the polymerase chain reaction. Proc Natl Acad Sci USA 1989; 86:3833-3837.
36. Burton D R, Barbas C F 3rd, Persson M A, Koenig S, Chanock R M, Lerner R A: A large array of human monoclonal antibodies to type 1 human immunodeficiency virus from combinatorial libraries of asymptomatic seropositive individuals. Proc Natl Acad Sci USA 1991; 88(22):10134-10137.
37. Burton D R, Pyati J, Koduri R, Sharp S J, Thornton G B, Parren P W, et al.: Efficient neutralization of primary isolates of HIV-1 by a recombinant human monoclonal antibody. Science 1994; 266 (5187):1024-1027.
38. Roben P, Moore J P, Thali M, Sodroski J, Barbas C F 3rd, Burton D R: Recognition properties of a panel of human recombinant Fab fragments to the CD4 binding site of gp120 that show differing abilities to neutralize human immunodeficiency virus type 1. J Virol 1994; 68(8):4821-4828.
39. Muster T, Steindl F, Purtscher M, Trkola A, Klima A, Himmler G, et al.: A conserved neutralizing epitope on gp41 of human immunodeficiency virus type 1. J Virol 1993; 67(11):6642-6647.
40. Zwick M B, Labrijn A F, Wang M, Spenlehauer C, Saphire E O, Binley J M, et al.: Broadly neutralizing antibodies targeted to the membrane-proximal external region of human immunodeficiency virus type 1 μlycoprotein gp41. J Virol 2001; 75(22):10892-10905.
41. Trkola A, Purtscher M, Muster T, Ballaun C, Buchacher A, Sullivan N, et al.: Human monoclonal antibody 2G12 defines a distinctive neutralization epitope on the gp120 μlycoprotein of human immunodeficiency virus type 1. J Virol 1996; 70(2):1100-1108.
42. Scanlan C N, Pantophlet R, Wormald M R, Ollmann Saphire E, Stanfield R, Wilson I A, et al.: The broadly neutralizing anti-human immunodeficiency virus type 1 antibody 2G12 recognizes a cluster of alpha1—>2 mannose residues on the outer face of gp120. J Virol 2002; 76(14):7306-7321.
43. Binley J M, Wrin T, Korber B, Zwick M B, Wang M, Chappey C, et al.: Comprehensive cross-clade neutralization analysis of a panel of anti-human immunodeficiency virus type 1 monoclonal antibodies. J Virol 2004; 78(23):13232-13252.
44. Mehandru S, Wrin T, Galovich J, Stiegler G, Vcelar B, Hurley A, et al.: Neutralization of newly transmitted human immunodeficiency virus type 1 by monoclonal antibodies 2G12, 2F5 and 4E10. J Virol 2004; 78:14039-14042.
45. Gray E S, Meyers T, Gray G, Montefiori D C, Morris L: Insensitivity of Paediatric HIV-1 Subtype C Viruses to Broadly Neutralising Monoclonal Antibodies Raised against Subtype. B PLoS Med 2006; 18; 3(7):e255.

1. An isolated antibody, or one of its functional fragments, said antibody or one of its said fragments being capable of binding specifically to the R7V epitope (RTPKIQV—SEQ ID No 11) and capable of neutralizing HIV strains, wherein it comprises:
i) a light chain comprising the complementarity determining regions CDRs comprising amino acid sequence SEQ ID No 1 (QSVLYSSNNKNY), SEQ ID No 2 (WAS) and SEQ ID No 3 (QQYYSTPQT), or CDRs which sequences have at least 80%, preferably 90% identity, after optimum alignment, with the sequence SEQ ID No 1, 2 or 3, and
ii) a heavy chain comprising the CDRs comprising amino acid sequence SEQ ID
No 6 (GGSISSYY), SEQ ID No 7 (IYYSGST) and SEQ ID No 8 (ARGRSWFSY), or CDRs whose sequence have at least 80%, preferably 90% identity, after optimum alignment, with the sequence SEQ ID No 6, 7 and 8.

2. The antibody according to claim 1 which is a fully human monoclonal antibody or functional fragments thereof.

3. The antibody according to claim 1 or 2 claim 1, wherein it comprises a light chain comprising an amino acid sequence having at least 80%, preferably 90% identity, after optimum alignment, with the amino acid sequence displayed in FIG. 3B—SEQ ID No 4 or a light chain encoded by a nucleotidic sequence comprising the sequence as depicted in FIG. 3A—SEQ ID No 5 or a sequence having at least 80%, preferably 25 90% identity, after optimum alignment, with SEQ ID No 5.

4. The antibody according to claim 1, wherein it comprises a heavy chain comprising an amino acid sequence having at least 80%, preferably 90% identity, after optimum alignment, with the amino acid sequence displayed in in FIG. 3D—SEQ ID No 9 or a heavy chain encoded by a nucleotidic sequence comprising the sequence as depicted in FIG. 3C—SEQ ID No 10 or a sequence having at least 80%, preferably 90% identity, after optimum alignment, with SEQ ID No 10.

5. The antibody according to claim 1 comprising a light chain comprising the amino acid sequence displayed in FIG. 3B—SEQ ID No 4 and a heavy chain comprising the amino acid sequence displayed in FIG. 3D—SEQ ID No 9, or functional fragments thereof chosen from of Fv, scFv, Fab, F(ab′)₂, F(ab′), scFv-Fc type or diabodies.

6. An isolated nucleic acid comprising a sequence having at least 80%, preferably 90% identity after optimum alignment with the sequence SEQ ID No. 5.

7. An isolated nucleic acid comprising a sequence having at least 80%, preferably 90% 10 identity after optimum alignment with the sequence SEQ ID No. 10.

8. A vector comprising a nucleic acid as defined in claim 6.

9. A baculovirus transfer vector comprising the nucleic acid sequence as defined in claim 6.

10. A host cell transformed by or comprising a vector according to claim 8.

11. A host cell according to claim 10 wherein it is an insect cell, such as Sf9 cells, a bacterial cell, a yeast cell, an animal cell, in particular a mammalian cell, such as EBV immortalized B lymphocytes, CHO, genetically modified CHO to produce low fucosylated antibodies, or YB2/0.

12. A method of producing of an antibody as defined in claim 1, or one of its functional fragments thereof, comprising the steps of:
a) culturing in a medium and appropriate culture conditions a host cell; and
b) extracting said antibodies from the culture medium of said cultured cells.

13. An antibody as defined in claim 1, or one of its functional fragments, as a medicament.

14. A pharmaceutical composition comprising the antibody as defined in claim 1, or one of its functional thereof, and an excipient and/or a pharmaceutically acceptable vehicle.

15. A combination product for simultaneous, separate or sequential use, comprising at least one agent currently used in treatment of AIDS and the antibody according to claim 1.

16. The method of using an antibody according to claim 1 for treating HIV infection, AIDS, for example in patients under HAART treatment and in particular in patients in failure of HAART treatment.

17. A baculovirus transfer vector comprising the nucleic acid sequence as defined in claim 7.


SEQUENCE LISTING (51 sequences)

SEQ ID NO: 1 - protein, 12 aa, Artificial sequence
  Complementary determining region (CDR)
QSVLYSSNNKNY

SEQ ID NO: 2 - protein, 3 aa, Artificial sequence
  Complementary determining region (CDR)
WAS

SEQ ID NO: 3 - protein, 9 aa, Artificial sequence
  Complementary determining region (CDR)
QQYYSTPQT

SEQ ID NO: 4 - protein, 135 aa, Artificial sequence
  Variable region of light (K4) chain (Fig. 3B)
LWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPQTFGQGTKLEIKRTVAAPSVFIFPPS

SEQ ID NO: 5 - DNA, 405 bp, Artificial sequence
  Variable region of light (K4) chain (Fig. 3A)
ctctggatctctggtgcctacggggacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctcagacttttggccaggggaccaagctggagatcaaacgaactgtggctgcaccatctgtcttcatcttcccgccatcg

SEQ ID NO: 6 - protein, 8 aa, Artificial sequence
  CDR
GGSISSYY

SEQ ID NO: 7 - protein, 7 aa, Artificial sequence
  CDR
IYYSGST

SEQ ID NO: 8 - protein, 9 aa, Artificial sequence
  CDR
ARGRSWFSY

SEQ ID NO: 9 - protein, 129 aa, Artificial sequence
  Variable region of heavy (M4) chain (Fig. 3D)
PRWVLSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARGRSWFSYWGQGTLVTVSSGSASAPTL

SEQ ID NO: 10 - DNA, 388 bp, Artificial sequence
  Variable region of heavy (M4) chain (Fig. 3C)
cccagatgggtcctgtcccaggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgccgcagacacggccgtgtattactgtgcgagaggccgttcgtggtttagctactggggccagggaaccctggtcaccgtctcctcagggagtgcatccgccccaacccttt

SEQ ID NO: 11 - protein, 7 aa, Artificial sequence
  R7V epitope
RTPKIQV

SEQ ID NO: 12 - DNA, 72 bp, Artificial sequence
  pVT-Ck (Fig. 1A)
atggacatgcgtgtgcccgctcaactcctgggcctgctgctgctctggctcccaggtgcgcgctgtcgtacg

SEQ ID NO: 13 - protein, 24 aa, Artificial sequence
  pVT-Ck (Fig. 1A)
MDMRVPAQLLGLLLLWLPGARCRT

SEQ ID NO: 14 - DNA, 63 bp, Artificial sequence
  pVT-C?1 (Fig. 1B)
atggagttcggcctgagctggctgttcctggtggctattcttaagggtgtccagtgtgctagc

SEQ ID NO: 15 - protein, 21 aa, Artificial sequence
  pVT-C?1 (Fig. 1B)
MEFGLSWLFLVAILKGVQCAS

SEQ ID NO: 16 - DNA, 62 bp, Artificial sequence
  Primer FOR-M4
ccatcttaagggtgtccagtgtcaggtgcagctgcaggagtcgggcccaggactggtgaagc

SEQ ID NO: 17 - DNA, 31 bp, Artificial sequence
  Primer BAC-M4
gcatgctagctgaggagacggtgaccagggt

SEQ ID NO: 18 - DNA, 34 bp, Artificial sequence
  Primer FOR-K4
cgatgcgcgctgtgacatcgtgatgacccagtct

SEQ ID NO: 19 - DNA, 37 bp, Artificial sequence
  Primer BAC-K4
cgatcgtacgtttgatctccagcttggtcccctggcc

SEQ ID NO: 20 - DNA, 17 bp, Artificial sequence
  Primer hVH1/VH7
atggactggacctggag

SEQ ID NO: 21 - DNA, 18 bp, Artificial sequence
  Primer hVH2
atggacatactttgttcc

SEQ ID NO: 22 - DNA, 18 bp, Artificial sequence
  Primer hVH3
atggagtttgggctgagc

SEQ ID NO: 23 - DNA, 17 bp, Artificial sequence
  Primer hVH4
atgaaacacctgtggtt

SEQ ID NO: 24 - DNA, 18 bp, Artificial sequence
  Primer hVH5
atggggtcaaccgccatc

SEQ ID NO: 25 - DNA, 18 bp, Artificial sequence
  Primer hVH6
atgtctgtctccttcctc

SEQ ID NO: 26 - DNA, 23 bp, Artificial sequence
  Primer hCG
ggaagtagtccttgaccaggcag

SEQ ID NO: 27 - DNA, 21 bp, Artificial sequence
  Primer hCM
ggagacgagggggaaaagggt

SEQ ID NO: 28 - DNA, 22 bp, Artificial sequence
  Primer hVL1a
tcactgcacaggstccwgggcc

SEQ ID NO: 29 - DNA, 22 bp, Artificial sequence
  Primer hVL1b
tcactgtgcagggtcctgggcc

SEQ ID NO: 30 - DNA, 20 bp, Artificial sequence
  Primer hVL2
ctcctcactcaggrcacagg

SEQ ID NO: 31 - DNA, 19 bp, Artificial sequence
  Primer hVL3a
ctcctcactytctgcacag

SEQ ID NO: 32 - DNA, 19 bp, Artificial sequence
  Primer hVL3b
ctcctctctcactgcacag

SEQ ID NO: 33 - DNA, 20 bp, Artificial sequence
  Primer hVL3c
tccttgcttactgcacagga

SEQ ID NO: 34 - DNA, 23 bp, Artificial sequence
  Primer hVL3d
tcactctttgcataggttctgtg

SEQ ID NO: 35 - DNA, 21 bp, Artificial sequence
  Primer hVL4
ctcctcctccactgsacaggg

SEQ ID NO: 36 - DNA, 20 bp, Artificial sequence
  Primer hVL5
ttcctctctcactgcacagg

SEQ ID NO: 37 - DNA, 19 bp, Artificial sequence
  Primer hVL6
ctcctcgctcactgcacag

SEQ ID NO: 38 - DNA, 21 bp, Artificial sequence
  Primer hVL7
ctcctcactygctgcccaggg

SEQ ID NO: 39 - DNA, 20 bp, Artificial sequence
  Primer hVL8
ctccttgsttatggrtcagg

SEQ ID NO: 40 - DNA, 21 bp, Artificial sequence
  Primer hVL9
ctcctcagtctcctcacaggg

SEQ ID NO: 41 - DNA, 17 bp, Artificial sequence
  Primer hVL10
ctcctcactcactctgc

SEQ ID NO: 42 - DNA, 27 bp, Artificial sequence
  Primer hCLa
ctcagaggagggcgggaacagagtgac

SEQ ID NO: 43 - DNA, 27 bp, Artificial sequence
  Primer hCLb
ctcagaggacggcaggaacagagtgac

SEQ ID NO: 44 - DNA, 19 bp, Artificial sequence
  Primer hVK1
tcagctcctggggctyctg

SEQ ID NO: 45 - DNA, 21 bp, Artificial sequence
  Primer hVK2a
ctggggctgctaatgctctgg

SEQ ID NO: 46 - DNA, 21 bp, Artificial sequence
  Primer hVK2b
ctggggctgctcctggtctgg

SEQ ID NO: 47 - DNA, 21 bp, Artificial sequence
  Primer hVK3
tcctgctactctggctcccag

SEQ ID NO: 48 - DNA, 19 bp, Artificial sequence
  Primer hVK4
tgctctggatctctggtgc

SEQ ID NO: 49 - DNA, 30 bp, Artificial sequence
  Primer hVK5
ctcctcctttggatctctgataccagggca

SEQ ID NO: 50 - DNA, 24 bp, Artificial sequence
  Primer hVK6
ctctgggttccagcctccaggggt

SEQ ID NO: 51 - DNA, 26 bp, Artificial sequence
  Primer hCK
gatggcgggaagatgaagacagatgg